(19) United States
(12) Plant Patent Application Publication
(10) Pub. No.: US 2013/0291253 P1
(43) Pub. Date: Oct. 31, 2013

(54) Phalaenopsis orchid plant named 'Eye Wonder'

(50) ;

(71) Applicant: FLORICULTURA, Heemskerk (NL)

(72) Inventor: René Schoone, Assendelft (NL)

(21) Appl. No.: 13/986,325

(22) Filed: Apr. 22, 2013

Related U.S. Application Data
(60) Provisional application No. 61/638,009, filed on Apr. 25, 2012.

(30) Foreign Application Priority Data
Apr. 26, 2012 (NL) OPS905

(51) Int. Cl. A01H 5/00

(52) U.S. Cl. PLT/311

(57) ABSTRACT

A new and distinct Phalaenopsis plant named ‘Eye Wonder’ particularly characterized by flowers which are light purple with a yellow/green haze; plants which may be propagated economically and uniformly using tissue culture; plants which produce more than one inflorescence; long and sturdy inflorescences; and relatively short, dark-green foliage.

### LATIN NAME OF THE GENUS AND SPECIES OF THE PLANT CLAIMED

Phalaenopsis hybrid

### VARIETY DENOMINATION

‘Eye Wonder’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Eye Wonder’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Eye Wonder’ is a product of a controlled breeding program conducted by the inventor, Rene Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Eye Wonder’ originated from a cross made by the inventor in 1999 in Strengweg, Heemskerk, The Netherlands. The female or seed parent is the Phalaenopsis cultivar designated ‘Romance Valentine’, unpatented. The male or pollen parent is the Phalaenopsis cultivar designated ‘Barbara Buddha’, unpatented. The new Phalaenopsis ‘Eye Wonder’ was discovered and selected by the inventor as a single flowering plant within the progeny of the stated cross in a controlled environment in 2007 in Strengweg, Heemskerk, The Netherlands.

Asexual reproduction of the new Phalaenopsis cultivar by tissue culture was first performed in November, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Eye Wonder’, which in combination distinguish this Phalaenopsis as a new and distinct cultivar:

1. flowers which are light purple with a yellow/green haze;

2. plant produces more than one inflorescence;

3. plants may be propagated economically and uniformly using tissue culture;

4. inflorescences are long and sturdy; and

5. relatively short, dark-green foliage.

In comparison with the parental cultivars of ‘Eye Wonder’, the female parent ‘Romance Valentine’ has dark pink flowers and the male parent ‘Barbara Buddha’ has yellow/bronze colored flowers, whereas the flowers of ‘Eye Wonder’ are light purple with a yellow/green haze.

Presently, there is no other commercial cultivar to which ‘Eye Wonder’ can be meaningfully compared.

### BRIEF DESCRIPTION OF THE PHOTOGRAPHS

The accompanying photographs illustrate the overall appearance of the new Phalaenopsis ‘Eye Wonder’ showing the colors as true as is reasonably possible with colored reproductions of this type. Colors in the photographs may differ slightly from the color values cited in the detailed botanical description, which accurately describe the color of ‘Eye Wonder’.

FIG. 1 shows a side view perspective of a typical flowering plant of ‘Eye Wonder’ in a 12 cm pot, at 16 months of age.

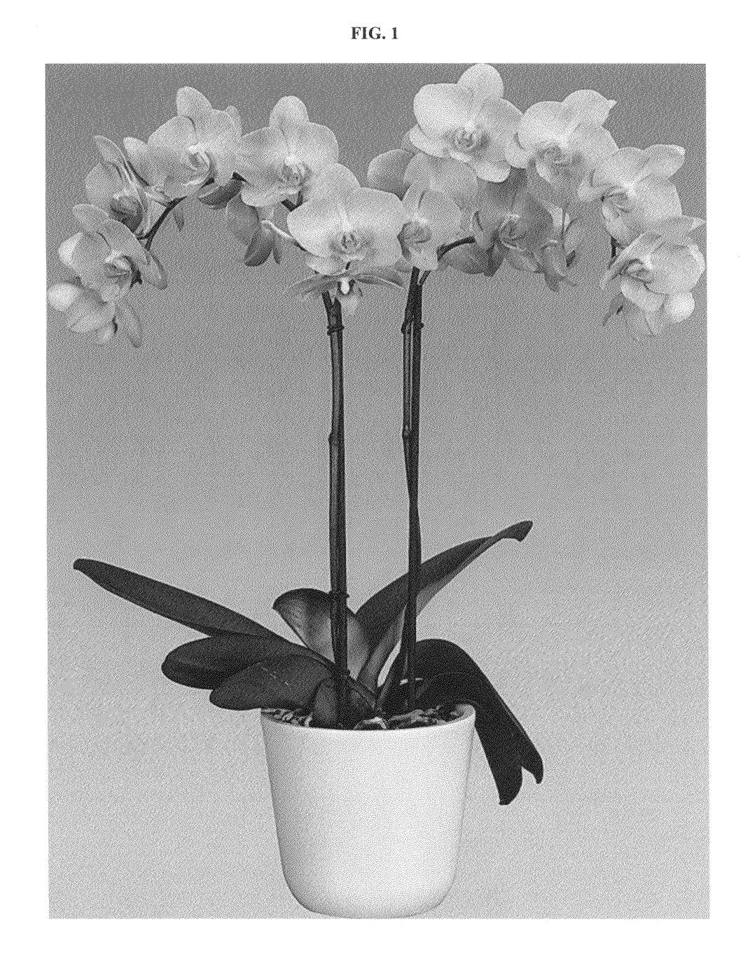

FIG. 2 shows a close-up view of the typical buds and flowers of ‘Eye Wonder’.

FIG. 3 shows a close-up view of the typical leaves of ‘Eye Wonder’.

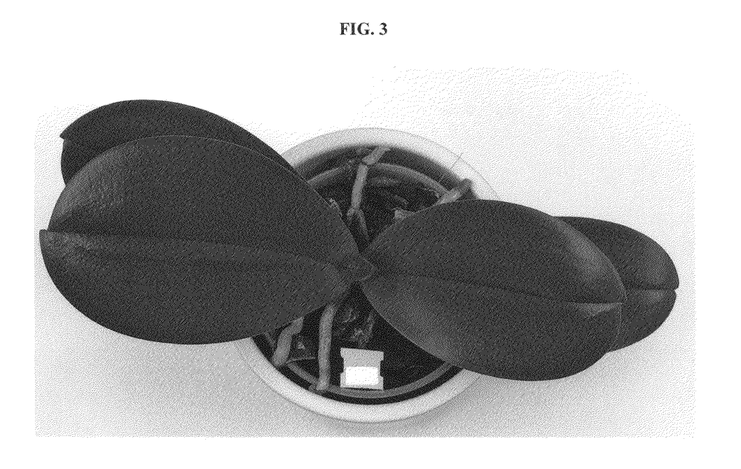

### DETAILED BOTANICAL DESCRIPTION

The new Phalaenopsis cultivar ‘Eye Wonder’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Eye Wonder’ as grown in a greenhouse in Strengweg, Heemskerk, The Netherlands, under conditions which closely approximate those generally used in commercial practice. Initially, the ideal temperature to grow plants of ‘Eye Wonder’ is 27° C. during the day and at night. Then, during the flowering phase of ‘Eye Wonder’, the ideal growing temperature is 20-22° C. during the day and 18° C. at night. Light levels for growing ‘Eye Wonder’ are a minimum of 5,000 lux and a maximum of 10,000 lux. A balanced fertilizer with level of 200 ppm N, 87 ppm P, 168 ppm K is applied. Duration of growth of ‘Eye Wonder’ from potting size is between 10 and 14 months.

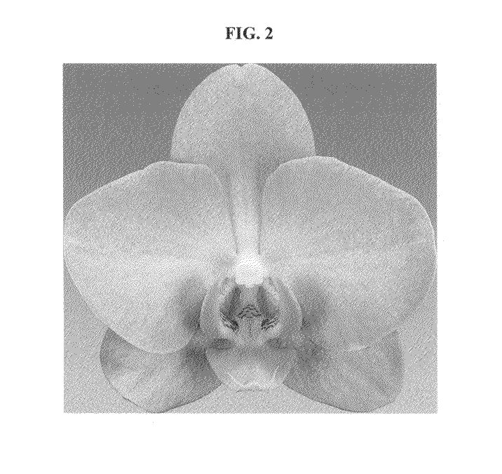

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Eye Wonder’ plants described is 12 months after potting.
Classification:
   Botanical.—Phalaenopsis hybrid.
Parentage:
   Female or seed parent.—Phalaenopsis cultivar designated ‘Romance Valentine’, unpatented.
   Male or pollen parent.—Phalaenopsis cultivar designated ‘Barbara Buddha’, unpatented.
Propagation:
   Type.—tissue culture.
   Rooting habit and description.—Fleshy; approximately 3 mm-7 mm wide and green in color (RHS 148D); freely branching. It takes 12 weeks for plants growing in tissue culture to initiate roots.
Plant:
   Size at maturity.—Height: about 60 cm Spread: about 60 cm.
   Growth habit.—standard; dark green leaves and a relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, the tip is blunt and asymmetric.
   Texture (upper & underside).—smooth and leathery.
   Pubescence.—3 to 4 pairs of leaves.
   Mature leaf length.—about 15 to 24 cm.
   Mature leaf width.—about 6 to 10 mm.
   Mature leaf thickness.—about 1,5 mm.
   Mature leaf color.—upper side: green (RHS 137A), parts of the edges are green (RHS 137C/RHS 137B). Under side: yellow/green (RHS 146B).
   Leaf base.—acute.
   Margin.—entire.
   Venation.—Pattern: parallel Color of midvein: upper side: green (RHS 139A) under side: yellow/green (RHS 145B).
Raceme:
   Quantity per plant.—about 1 to 2.
   Number of flowers per raceme.—about 7 to 12.
   Length.—about 49 to 55 cm.
   Diameter.—about 4 mm.
   Strength.—strong.
   Aspect.—upright.
   Texture.—glabrous and smooth.
   Color.—green (RHS N137D) which runs into RHS 137D.
   Internode.—Length: about 35 to 43 mm.
Inflorescence description:
   Appearance.—upright to slightly pendant, racemose inflorescence with bilaterally symmetrical flowers that open in succession beginning with the lowermost flower.
   Buds.—Height (from base to tip): about 21 mm to 26 mm Diameter (at midpoint): about 15 mm to 21 mm Shape: egg-shaped Color: yellow/green (RHS 154A) which runs into purple (RHS 72A).
   Flowering time.—For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
   Flowering longevity.—On the plant: about 4 to 6 months; lastingness of cut flowers: has not been observed.
   Fragrance.—no fragrance.
   Flower.—Rate of opening: Flowers fully opened about 2 to 3 days after petal and sepal separation. Orientation at opening: slanted upward and outward. Shape: see FIG. 2 Size: Height: about 56 mm to 69 mm Diameter: about 65 mm to 78 mm Depth of tube: about 10 mm.
   Petals.—Quantity and arrangement: six petals that are trimerous, overlapping and arranged in 2 whorls. Petals are more pronounced than sepals Arrangement: Inner whorl of petals comprises 3 petals, 2 lateral petals and labellum. 2 lateral petals: Overall shape: broadly ovate, little triangular and weakly cupped Apex: oval, a little flat and weakly undulate Margin: entire and weakly undulate Base: broadly ovate Length: about 35 mm Width: about 33 mm Texture: Upper surface: smooth and satiny Under surface: smooth and satiny Color (when fully opened): upper side: main color is purple (RHS N78D). At base and the edges white (RHS N155B), with a light yellow/green haze (RHS 145D) and spots at the base (RHS N78C). Under surface: main color is purple (RHS N78D/RHS N78C). From base and in center yellow/green (RHS 145D). Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire and weakly undulate Apex: oval Length: about 22 mm Width (not flattened): about 18 mm Texture: Upper and under surface: smooth and satiny Color (when fully opened): Midlobe: upper surface: main color is purple (RHS N78C. From base and on the sides orange/red (RHS N34D) and at the base a little yellow (RHS 13A). Under surface: main color is purple (N78D) with a white haze in the center (RHS N155B). On sides orange/red (RHS N34D) and very edge RHS N34A. Lateral lobes: upper surface: white (RHS NN155B) with yellow (RHS 13A) and purple/red spots and stripes (RHS 60A). Lower edge is orange/red (RHS N34D). At the top of the lob purple (RHS N78B). Under surface: base is white (RHS N155B) which runs into purple (RHS N78C). Lower edge is orange/red (RHS N34A) with yellow (RHS 13C). Chirri: about 7 mm. color: from purple (RHS N78D) to white (RHS N155C) to yellow (RHS 13D) Pestle (Callosities): Length: about 6 mm Width (not flattened): about 5 mm Color: main color is white (NN155C). Top and sides are yellow (RHS 13A). Spots are RHS red/purple (RHS 60A).
   Sepals.—Arrangement: Outer whorl comprises 3 sepals. Overall shape: elliptical and weakly cupped Margin: entire and weakly undulate Length: about 36 mm Width: about 27 mm Apex: oval; lateral: small point, dorsal: small notch Texture: Upper and under surface: smooth and satiny Color (when fully opened): main color dorsal and lateral is purple (RHS N78D). At base dorsal sepal some small purple spots (RHS 77D) and in center a green haze (RHS 145C). At base lateral sepal white (RHS N155B) which runs into purple (RHS 77D) with spots (RHS N78A). Under surface: main color dorsal sepals is purple (RHS N78B) with in the center a light yellow/green haze (RHS 145A).Main color and edges lateral sepals is purple (RHS N77B). At the base in the center a yellow/green haze (RHS 145B).
Reproductive organs:
   Arrangement.—The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
   Column.—Length: about 11 mm Diameter: about 7 mm Color: white (RHS NN155C) with purple (RHS N78D).
   Pollinia.—Quantity: two Diameter: about 1 mm Color: orange (RHS 26A).
   Ovary.—Length: about 5 mm Diameter: about 5 mm Color: white (RHS N155B).
   Pedicel.—Length: about 38 mm Diameter: about 4 mm Texture: glabrous and smooth Color: at the base yellow/green (RHS 146A, in the middle yellow/green (RHS 146D) and close to flower yellow/green (RHS 145D).
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Eye Wonder’, as illustrated and described herein.
